(19) United States
(12) Patent Application Publication
(10) Pub. No.: US 2026/0026742 A1
(43) Pub. Date: Jan. 29, 2026

(54) ELECTRONIC DEVICE AND METHOD OF DETECTING SLEEP STAGE

(71) Applicant: Wistron Corporation, New Taipei City (TW)

(72) Inventors: Yu-Hsuan Ho, New Taipei City (TW); Zong-Lin Tsai, New Taipei City (TW); Yu-Wen Huang, New Taipei City (TW); Shang-Min Yeh, New Taipei City (TW)

(73) Assignee: Wistron Corporation, New Taipei City (TW)

(21) Appl. No.: 18/830,585

(22) Filed: Sep. 11, 2024

(30) Foreign Application Priority Data
Jul. 23, 2024 (TW) 113127566

(51) Int. Cl. A61B 5/00; A61B 5/0205

(52) U.S. Cl. CPC A61B 5/4812; A61B 5/0205; A61B 5/7257

(57) ABSTRACT

The disclosure provides an electronic device and a method of detecting a sleep stage. The method includes the following. A radar signal is received, and a physiological signal is extracted from the radar signal. Fast Fourier transform is performed on the physiological signal by using a first window to obtain a transformed signal. A peak area ratio corresponding to the transformed signal is obtained according to a peak of the transformed signal. A first prediction result of the sleep stage is generated according to the peak area ratio by using a first machine learning model. The first prediction result is outputted.

### CROSS-REFERENCE TO RELATED APPLICATION

This application claims the priority benefit of Taiwan application serial no. 113127566, filed on Jul. 23, 2024. The entirety of the above-mentioned patent application is hereby incorporated by reference herein and made a part of this specification.

### BACKGROUND

### Technical Field

The disclosure relates to a signal detection technology, and more particularly, to an electronic device and a method of detecting a sleep stage.

### Description of Related Art

With the development of sleep medicine, people are paying more and more attention to sleep quality. Although there are currently many products on the market for monitoring a sleep status of a subject, the products are still unable to accurately detect a sleep stage that the subject enters. In order to accurately record the time when the subject enters each of the sleep stages, polysomnography (PSG) is required to be performed on the subject in a sleep laboratory. The above method is very inconvenient for the subject.

### SUMMARY

The disclosure provides an electronic device and a method of detecting a sleep stage, which may accurately determine the sleep stage that a subject enters.

An electronic device for detecting a sleep stage according to an embodiment of the disclosure includes a processor and a transceiver. The transceiver receives a radar signal. The processor is coupled to the transceiver. The processor is configured to extract a physiological signal from the radar signal, perform fast Fourier transform on the physiological signal by using a first window to obtain a transformed signal, obtain a peak area ratio corresponding to the transformed signal according to a peak of the transformed signal, generate a first prediction result of the sleep stage according to the peak area ratio by using a first machine learning model, and output the first prediction result through the transceiver.

A method of detecting a sleep stage according to an embodiment of the disclosure includes the following. A radar signal is received, and a physiological signal is extracted from the radar signal. Fast Fourier transform is performed on the physiological signal by using a first window to obtain a transformed signal. A peak area ratio corresponding to the transformed signal is obtained according to a peak of the transformed signal. A first prediction result of the sleep stage is generated according to the peak area ratio by using a first machine learning model. The first prediction result is outputted.

Based on the above, the electronic device and the method of detecting the sleep stage in the disclosure may generate the prediction result of the sleep stage that is more accurate and easy to interpret. The prediction result may indicate that the subject enters the sleep stages such as a wakefulness stage, a rapid eye movement (REM) stage, a light sleep stage, or a deep sleep stage.

### BRIEF DESCRIPTION OF THE DRAWINGS

FIG. 1 is a schematic view of an electronic device for detecting a sleep stage according to an embodiment of the disclosure.

FIG. 2 is a flowchart of a first phase of a method of detecting a sleep stage according to an embodiment of the disclosure.

FIG. 3 is a schematic view of a transformed signal according to an embodiment of the disclosure.

FIG. 4 is a schematic view of a peak area ratio of a transformed signal of a respiratory signal according to an embodiment of the disclosure.

FIG. 5 is a schematic view of a peak area ratio of a transformed signal of a heart rate signal according to an embodiment of the disclosure.

FIG. 6 is a schematic view of a coupled signal of a respiratory signal according to an embodiment of the disclosure.

FIG. 7 is a schematic view of a coupled signal of a heart rate signal according to an embodiment of the disclosure.

FIG. 8 is a schematic view of an average signal according to an embodiment of the disclosure.

FIG. 9 is a schematic view of segmenting a sample from an average signal according to an embodiment of the disclosure.

FIG. 10 is a flowchart of a second phase of a method of detecting a sleep stage according to an embodiment of the disclosure.

FIG. 11 is a schematic view of correcting a first prediction result according to an embodiment of the disclosure.

FIG. 12 is a schematic view of a comparison between prediction results of sleep stages according to an embodiment of the disclosure.

FIG. 13 is a flowchart of a method of detecting a sleep stage according to an embodiment of the disclosure.

### DETAILED DESCRIPTION OF DISCLOSED EMBODIMENTS

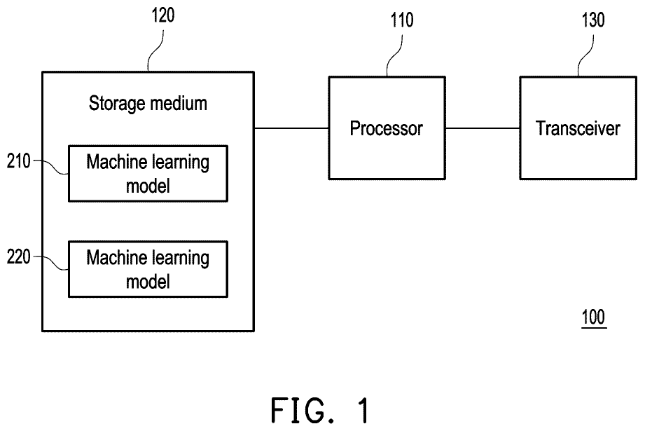

FIG. 1 is a schematic view of an electronic device 100 for detecting a sleep stage according to an embodiment of the disclosure. The electronic device 100 may include a processor 110, a storage medium 120, and a transceiver 130.

The processor 110 is, for example, a central processing unit (CPU), or other programmable general-purpose or special-purpose micro control units (MCU), microprocessors, digital signal processors (DSP), programmable controllers, application specific integrated circuits (ASIC), graphics processing units (GPU), image signal processors (ISP), image processing units (IPU), arithmetic logic units (ALU), complex programmable logic devices (CPLD), field programmable gate arrays (FPGA), or others similar elements or a combination of the above elements. The processor 110 may be coupled to the storage medium 120 and the transceiver 130, and access and execute multiple modules and various applications stored in the storage medium 120.

The storage medium 120 is, for example, any type of fixed or removable random access memory (RAM), read-only memory (ROM), flash memory, hard disk drive (HDD), solid state drive (SSD), or similar elements or a combination of the above elements, and is used to store the modules or various applications that may be executed by the processor 110. In an embodiment, the storage medium 120 may store a machine learning model 210 and a machine learning model 220.

The transceiver 130 transmits or receives a signal in a wireless or wired manner. The transceiver 130 may further perform operations such as low noise amplification, impedance matching, frequency mixing, upward or downward frequency conversion, filtering, amplification, and similar operations.

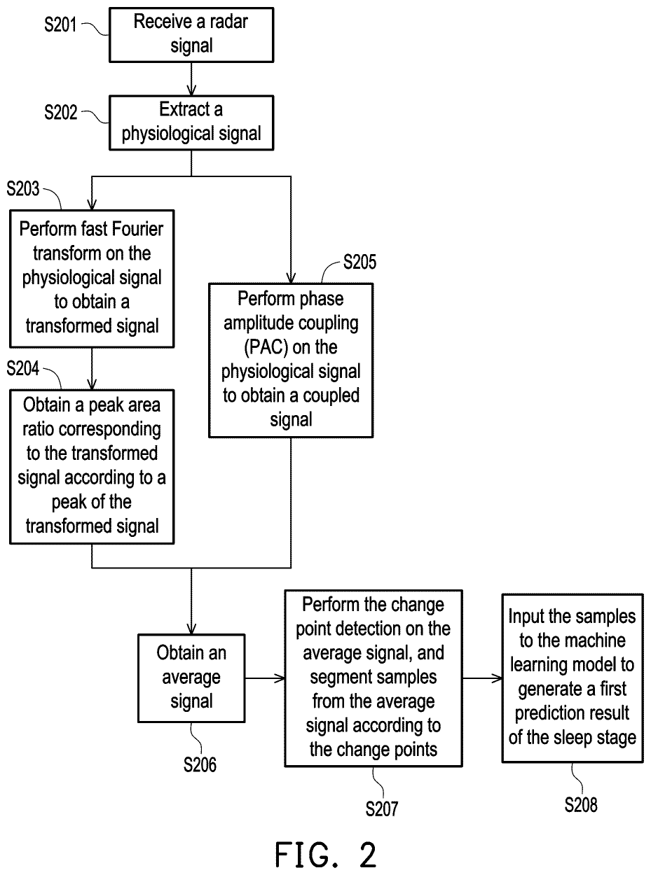

FIG. 2 is a flowchart of a first phase of a method of detecting a sleep stage according to an embodiment of the disclosure. The method may be implemented by the electronic device 100 shown in FIG. 1. In step S201, the processor 110 may receive a radar signal through the transceiver 130. For example, the processor 110 may be communicatively connected to a millimeter wave radar through the transceiver 130, transmit a radio frequency signal to a sleeping subject through the millimeter wave radar, and receive the radar signal through the millimeter wave radar. The radar signal is a reflection signal of the radio frequency signal.

In step S202, the processor 110 may extract a physiological signal of the subject from the radar signal. The physiological signal may include a heart rate (HR) signal, a heartbeat (HB) signal, or a respiratory signal. The respiratory signal may include stability, a respiratory rate (RR), or respiratory variability, etc. The processor 110 may extract the physiological signal from the radar signal based on, for example, a machine learning algorithm. The processor 110 may, for example, perform pre-processing, low-pass filtering, or high-pass filtering on the radar signal to extract the physiological signal.

In step S203, the processor 110 may perform fast Fourier transform (FFT) on the physiological signal by using a first window to obtain a transformed signal. The first window may be a time period. For example, the first window may be between 2 minutes and 4 minutes.

In step S204, the processor 110 may obtain a peak area ratio (PAR) or a peak centered area ratio corresponding to the transformed signal according to a peak of the transformed signal.

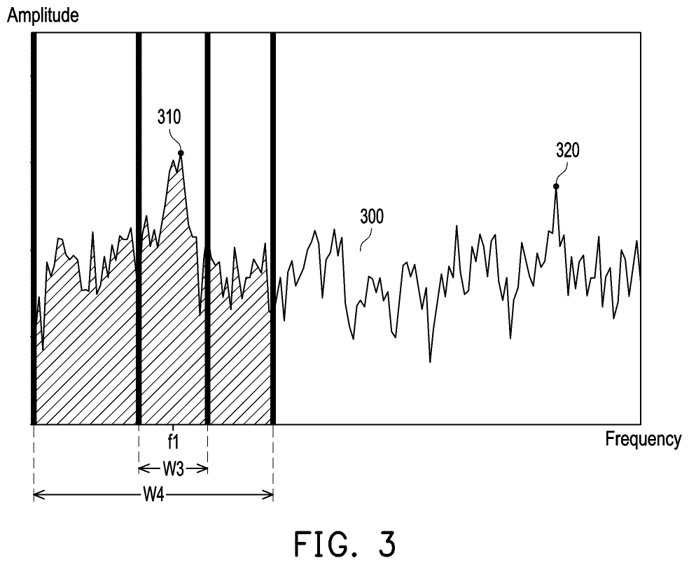

Specifically, the processor 110 may detect a maximum value of the transformed signal to obtain the peak of the transformed signal. FIG. 3 is a schematic view of a transformed signal 300 according to an embodiment of the disclosure. The processor 110 may detect a maximum value of the transformed signal 300 to obtain a peak 310. In an embodiment, the processor 110 may perform change point detection (CPD) on the transformed signal 300 to obtain one or more change points (e.g., change points 310 or 320), and select a change point with a maximum value from the one or more change points as the peak.

Then, the processor 110 may sample the transformed signal 300 according to a window W3 to obtain a first peak area (or an area under curve (AUC)). The window W3 may be a frequency band, and a center of the window W3 corresponds to a frequency f1 of the peak 310. The processor 110 may sample the transformed signal 300 according to a window W4 to obtain a second peak area. The window W4 may be a frequency band greater than the window W3, and the window W4 may include the window W3. In other words, the window W4 may include the frequency f1 of the peak 310. The processor 110 may calculate a ratio of the first peak area and the second peak area to obtain a peak area ratio of the transformed signal 300. The peak area ratio of the transformed signal 300 calculated based on the peak 310 is approximately 0.235.

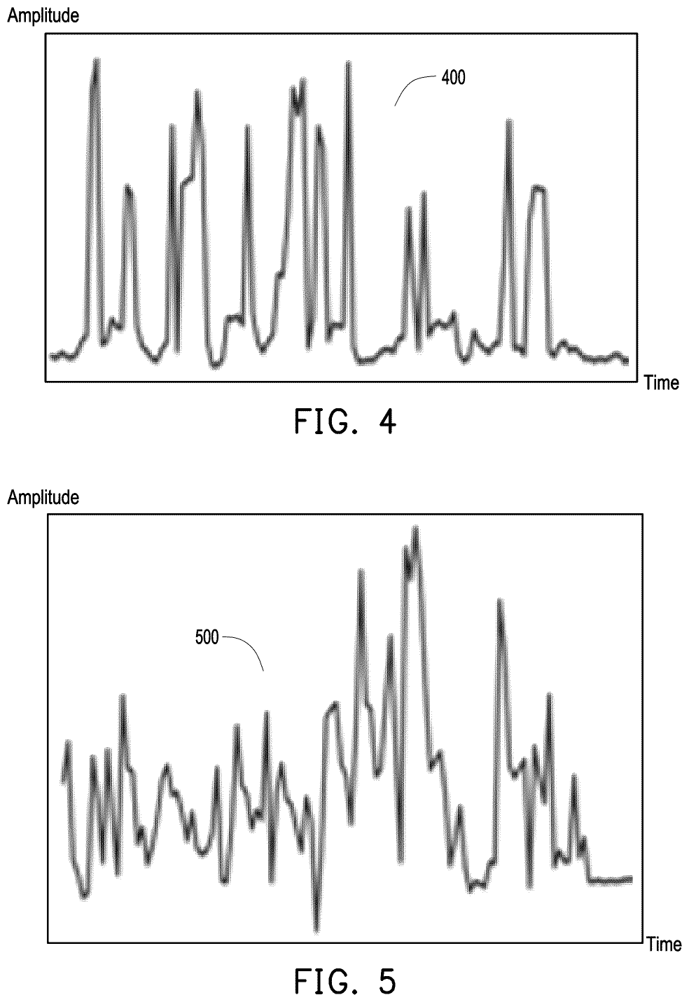

After step S201 to step S204 are repeatedly executed multiple times, the processor 110 may obtain multiple peak area ratios respectively corresponding to multiple time periods. The processor 110 may illustrate curves of the peak area ratios according to the peak area ratios, such as a peak area ratio 400 of the transformed signal of the respiratory signal shown in FIG. 4 or a peak area ratio 500 of the transformed signal of the heart rate signal shown in FIG. 5.

In an embodiment, the processor 110 may set the window W4 according to a preset time period. For example, the processor 110 may set a starting point of the window W4 at 0 Hz and an end point of the window W4 at 0.4 Hz based on the respiratory rate of the human of about 0 to 0.4 Hz.

In an embodiment, the processor 110 may set the window W4 according to the frequency f1 of the peak 310. For example, the processor 110 may use the frequency f1 as a center of the window W4 to set the window W4.

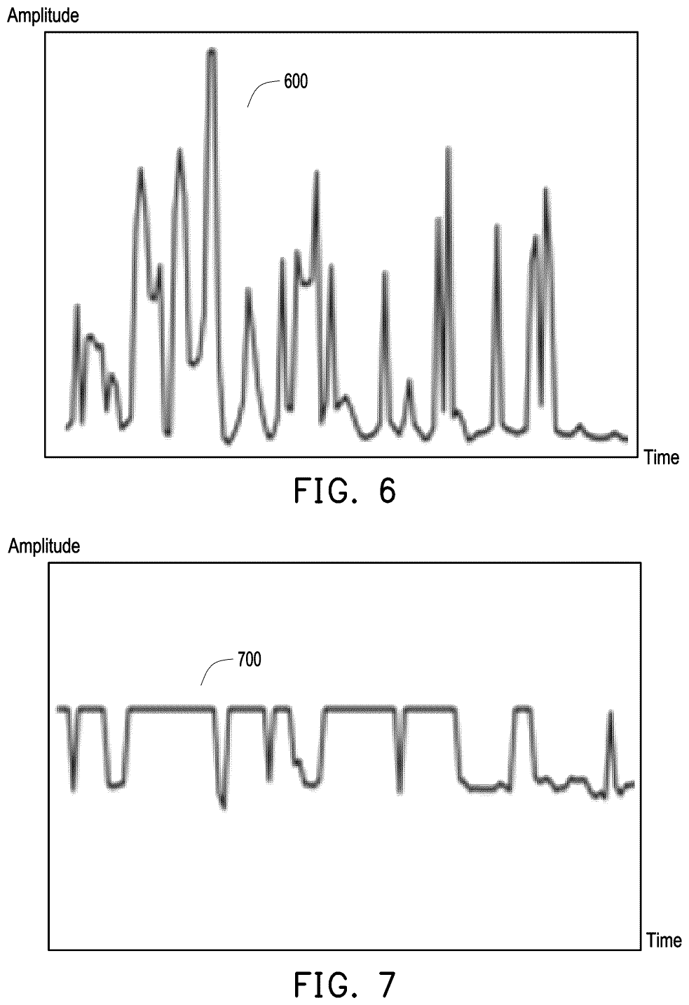

Returning to FIG. 2, in step S205, the processor 110 may perform phase amplitude coupling (PAC) on the physiological signal by using the first window to obtain a coupled signal, such as a coupled signal 600 of the respiratory signal as shown in FIG. 6 or a coupled signal 700 of the heart rate signal shown in FIG. 7.

In step S206, the processor 110 may obtain an average signal. Specifically, the processor 110 may perform normalization on a peak area ratio of the physiological signal to obtain a normalized peak area ratio, and may perform the normalization on a coupled signal of the physiological signal to obtain a normalized coupled signal. The processor 110 may calculate an average of the normalized peak area ratio and the normalized coupled signal to obtain the average signal.

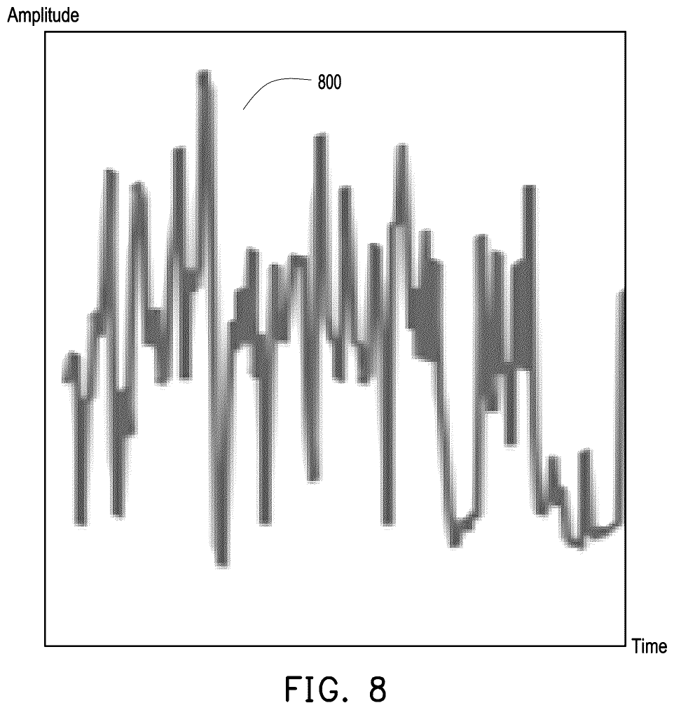

FIG. 8 is a schematic view of an average signal 800 according to an embodiment of the disclosure. For example, the processor 110 may perform the normalization on the transformed signal 400 of the respiratory signal, the transformed signal 500 of the heart rate signal, the coupled signal 600 of the respiratory signal, and the coupled signal 700 of the heart rate signal respectively to obtain the normalized transformed signal 400, the normalized transformed signal 500, the normalized coupled signal 600, and the normalized coupled signal 700. The processor 110 may calculate an average of the normalized transformed signal 400, the normalized transformed signal 500, the normalized coupled signal 600, and the normalized coupled signal 700 to obtain the average signal 800, as shown in FIG. 8.

Returning to FIG. 2, in step S207, the processor 110 may perform the change point detection on the average signal to obtain multiple change points, and segment one or more samples from the average signal according to the change points.

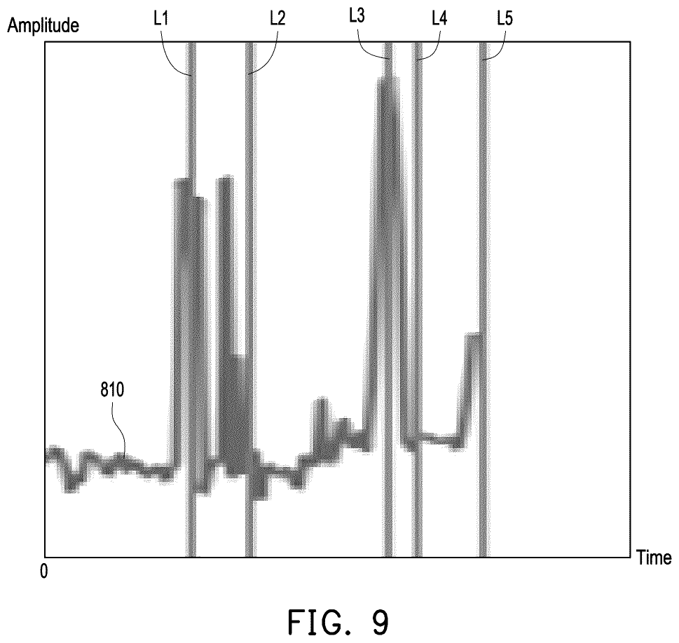

FIG. 9 is a schematic view of segmenting a sample from an average signal 810 according to an embodiment of the disclosure. The average signal 810 is, for example, a portion of the average signal 800. The processor 110 may perform the change point detection on the average signal 810 to obtain multiple change points. The change points correspond to a time point L1, a time point L2, a time point L3, a time point L4, and a time point L5 respectively. The processor 110 may segment multiple samples from the average signal 810 according to the time points, such as samples from a time period of 0 to L1, samples from a time period of L1 to L2, samples from a time period of L2 to L3, samples from a time period of L3 to L4, and samples from a time period of L4 to L5.

In step S208, the processor 110 may input the samples to the machine learning model 210 to generate a first prediction result of the sleep stage. The processor 110 may output the first prediction result through the transceiver 130 for user reference. The first prediction result may be used to indicate that the subject enters a wakefulness stage, a REM stage, or a non-rapid eye movement (NREM) stage. The NREM stage may include a stage N1, a stage N2, or a stage N3. The stage N1 and the stage N2 may be defined as light sleep stages, and the stage N3 may be defined as a deep sleep stage.

The machine learning model 210 may be a deep learning model, such as a convolutional neural network (CNN) model. The processor 110 may collect historical radar signals and generate multiple historical samples corresponding to the historical radar signals in a manner similar to steps S201 to step S207. The processor 110 may use the historical samples and multiple labels respectively corresponding to the historical samples as training data to train the machine learning model 210. For example, the processor 110 may train the machine learning model 210 according to the training data based on a supervised machine learning algorithm. The labels of the historical samples may be determined by the professional personnel or according to a result of PSG.

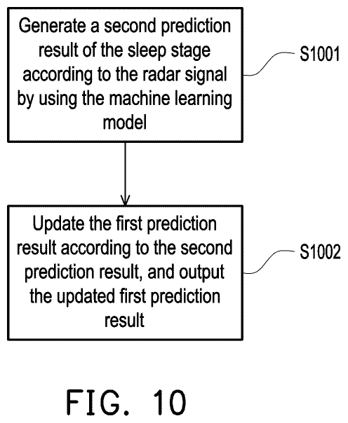

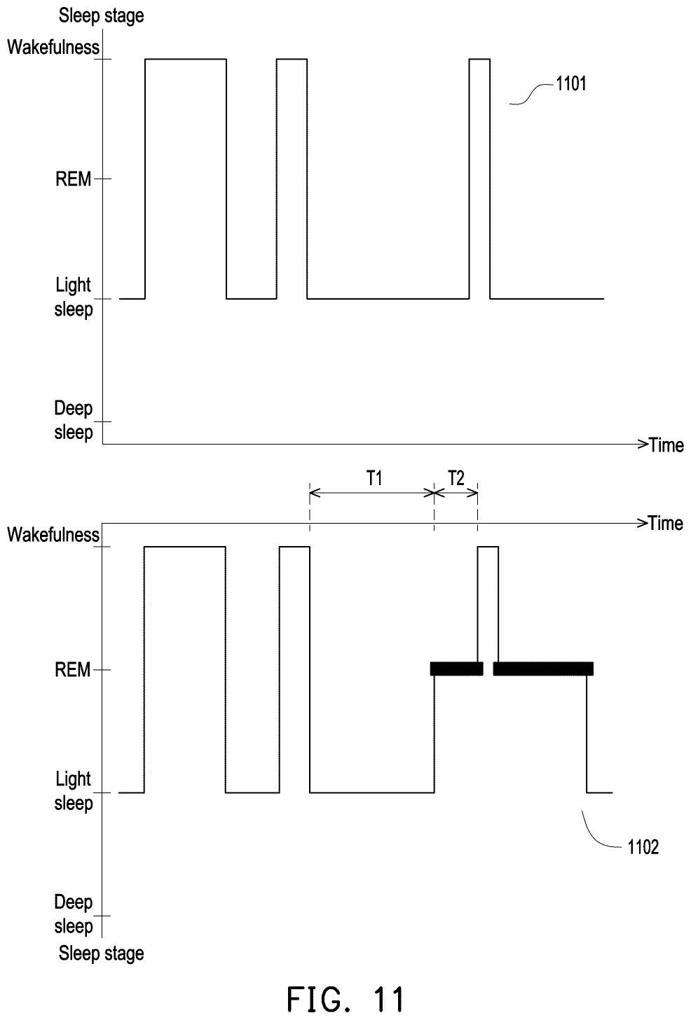

FIG. 10 is a flowchart of a second phase of a method of detecting a sleep stage according to an embodiment of the disclosure. The method may be implemented by the electronic device 100 shown in FIG. 1. In step S1001, the processor 110 may generate a second prediction result of the sleep stage according to the radar signal by using the machine learning model 220. In step S1002, the processor 110 may update the first prediction result according to the second prediction result, and output the updated first prediction result through the transceiver 130 for user reference.

Specifically, the processor 110 may extract one or more samples from raw data of the radar signal by using a second window. The second window may be shorter than the first window. For example, the second window may be shorter than 2 minutes. The processor 110 may input the samples to the machine learning model 220 to generate the second prediction result of the sleep stage. The second prediction result may be used to indicate that the subject enters the wakefulness stage, the REM stage, or the NREM stage. The NREM stage may include the stage N1, the stage N2, or the stage N3. The stage N1 and the stage N2 may be defined as the light sleep stages, and the stage N3 may be defined as the deep sleep stage.

The machine learning model 220 is, for example, a CNN model. The processor 110 may collect the historical radar signals and extract the historical samples from the radar signal by using the second window. The processor 110 may use the historical samples and the labels respectively corresponding to the historical samples as the training data to train the machine learning model 220. For example, the processor 110 may train the machine learning model 220 according to the training data based on the supervised machine learning algorithm. That is, the processor 110 may generate annotated answers based on PSG to perform training and verification on the machine learning model 220. The labels of the historical samples may be determined by the professional personnel or according to the result of PSG.

After obtaining the first prediction result and the second prediction result of the specific sample, the processor 110 may perform refinement on the first prediction result according to the second prediction result to update a portion of the first prediction result corresponding to the specific sample. The second prediction result may indicate multiple confidence levels respectively corresponding to multiple sleep stages. The processor 110 may update the portion of the first prediction result corresponding to the specific sample to the specific sleep stage in response to the second prediction result of the specific sample indicating that the confidence level of the specific sleep stage is greater than a threshold value. The threshold values of different sleep stages may be the same or different. For example, it is assumed that the threshold value of the light sleep stage is 0.5, and the second prediction result indicates that the probability that the specific sample is in the wakefulness stage, the REM stage, the light sleep stage, and the deep sleep stage is [0.1, 0.1, 0.7, 0.1] respectively. Since the second prediction result indicates that the probability of 0.7 that the specific sample is in the light sleep stage is greater than the threshold value of 0.5, the processor 110 may determine that the specific sample corresponds to the light sleep stage. Accordingly, the processor 110 may update the portion of the first prediction result corresponding to the specific sample to the light sleep stage.

FIG. 11 is a schematic view of correcting a first prediction result according to an embodiment of the disclosure. It is assumed that a first prediction result 1101 indicates that a portion of the radar signal during a period T1 corresponds to the light sleep stage, and a portion of the radar signal during a period T2 corresponds to the light sleep stage. If the second prediction result indicates that a confidence level of the portion of the radar signal during the period T1 corresponding to the light sleep stage is greater than the threshold value, the processor 110 may maintain the portion of the first prediction result 1101 during the period T1 in the light sleep stage. If the second prediction result indicates that the confidence level of the portion of the radar signal during the period T1 corresponding to a non-light sleep stage (e.g., the REM stage) is less than or equal to the threshold value, the processor 110 may maintain the portion of the first prediction result 1101 during the period T1 in the light sleep stage.

On the other hand, if the second prediction result indicates that a confidence level of the portion of the radar signal during the period T2 corresponding to the REM stage is greater than the threshold value, the processor 110 may modify the portion of the first prediction result 1101 during the period T2 from the light sleep stage to the REM stage, thereby generating an updated first prediction result 1102. The processor 110 may output the updated first prediction result 1102 through the transceiver 130 for user reference.

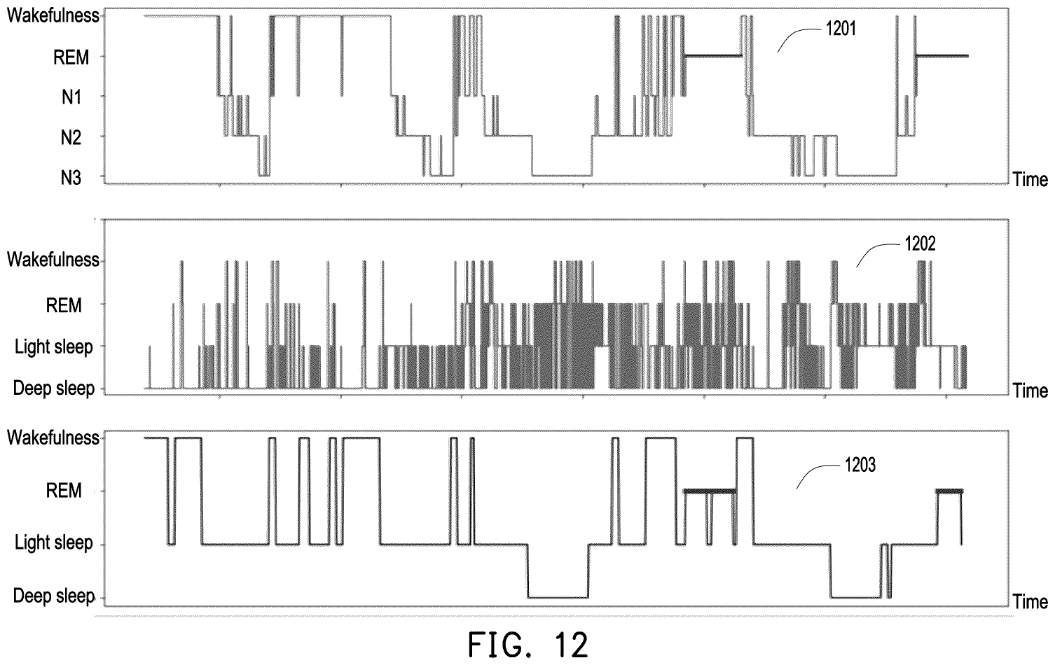

FIG. 12 is a schematic view of a comparison between prediction results of sleep stages according to an embodiment of the disclosure. A prediction result 1201 is a prediction result generated by PSG. A prediction result 1202 is a prediction result generated by a conventional method based on the machine learning algorithm. A prediction result 1203 is a prediction result generated by the electronic device 100. According to FIG. 12, compared to the prediction result 1202, the prediction result 1203 has better readability, and is more similar to the prediction result 1201. Therefore, the prediction result 1203 is better than the prediction result 1202.

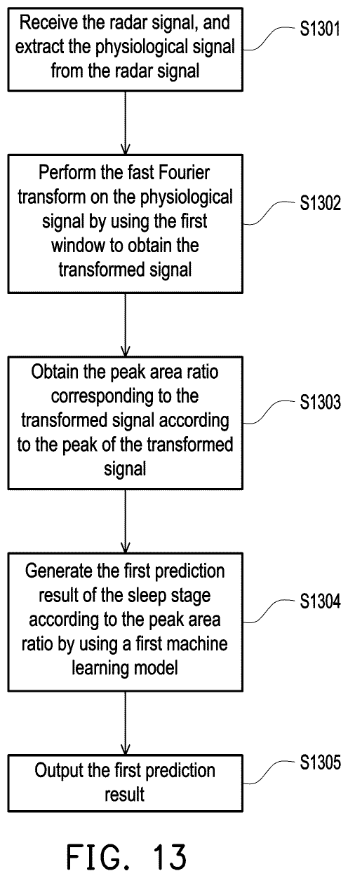

FIG. 13 is a flowchart of a method of detecting a sleep stage according to an embodiment of the disclosure. The method may be implemented by the electronic device 100 shown in FIG. 1. In step S1301, the radar signal is received, and the physiological signal is extracted from the radar signal. In step S1302, the fast Fourier transform is performed on the physiological signal by using the first window to obtain the transformed signal. In step S1303, the peak area ratio corresponding to the transformed signal is obtained according to the peak of the transformed signal. In step S1304, the first prediction result of the sleep stage is generated according to the peak area ratio by using a first machine learning model. In step S1305, the first prediction result is outputted.

Based on the above, compared to detection results of the sleep stage generated by the conventional machine learning model that are difficult for users to interpret, the electronic device in the disclosure may generate the prediction results of the sleep stage that are more accurate and easy to interpret. The electronic device may perform the pre-processing on the physiological signals extracted from the radar signal through a larger window through the methods such as the fast Fourier transform and the phase amplitude coupling, and generate the prediction result of the first phase according to the pre-processed physiological signal based on the first machine learning model. Within a fixed time period, the prediction result of the first phase is relatively stable and do not change frequently between the sleep stages, so the users may easily interpret the prediction result. Then, the electronic device may extract the raw data from the radar signal by using a smaller window to train a second machine learning model according to the raw data or generate the prediction result of the second phase by using the second machine learning model. The prediction result of the second phase is less stable and changes frequently between the sleep stages, so the prediction result of the second phase is more difficult to interpret. However, for a short time period, the prediction result of the second phase may be more accurate. The electronic device may correct the prediction result of the first phase by using the prediction result of the second phase, thereby generating the prediction result of the sleep stage that is accurate and easy to interpret.

What is claimed is:

1. An electronic device for detecting a sleep stage, comprising:
a transceiver receiving a radar signal; and
a processor coupled to the transceiver, wherein the processor is configured to:
extract a physiological signal from the radar signal;
perform fast Fourier transform on the physiological signal by using a first window to obtain a transformed signal;
obtain a peak area ratio corresponding to the transformed signal according to a peak of the transformed signal;
generate a first prediction result of the sleep stage according to the peak area ratio by using a first machine learning model; and
output the first prediction result through the transceiver.

2. The electronic device according to claim 1, wherein the processor is further configured to:
extract a sample of the radar signal by using a second window, wherein the second window is shorter than the first window;
input the sample into a second machine learning model to generate a second prediction result of the sleep stage; and
update a portion of the first prediction result corresponding to the sample according to the second prediction result.

3. The electronic device according to claim 2, wherein the processor is further configured to:
in response to the second prediction result indicating that a confidence level corresponding to a first sleep stage is greater than a threshold value, update the portion of the first prediction result to the first sleep stage.

4. The electronic device according to claim 1, wherein the processor is further configured to:
perform phase amplitude coupling on the physiological signal by using the first window to obtain a coupled signal; and
generate the first prediction result according to the coupled signal and the peak area ratio by using the first machine learning model.

5. The electronic device according to claim 4, wherein the processor is further configured to:
perform normalization on the peak area ratio to obtain the normalized peak area ratio;
perform the normalization on the coupled signal to obtain the normalized coupled signal;
calculate an average of the normalized peak area ratio and the normalized coupled signal to obtain an average signal; and
generate the first prediction result according to the average signal by using the first machine learning model.

6. The electronic device according to claim 5, wherein the processor is further configured to:
perform change point detection on the average signal to obtain a plurality of change points;
segment a sample from the average signal according to the change points; and
input the sample to the first machine learning model to generate the first prediction result.

7. The electronic device according to claim 1, wherein the processor is further configured to:
perform change point detection on the transformed signal to obtain the peak.

8. The electronic device according to claim 7, wherein the processor is further configured to:
sample the transformed signal according to a third window to obtain a first peak area, wherein a center of the third window corresponds to a frequency of the peak;
sample the transformed signal according to a fourth window to obtain a second peak area, wherein the fourth window comprises the frequency of the peak, and the fourth window is greater than the third window; and
calculate a ratio of the first peak area and the second peak area to obtain the peak area ratio.

9. The electronic device according to claim 8, wherein the processor is further configured to perform one of the following:
setting the fourth window according to a preset time period; and
setting the fourth window according to the frequency of the peak, wherein a center of the fourth window corresponds to the frequency.

10. The electronic device according to claim 1, wherein the physiological signal comprises at least one of a heart rate signal and a respiratory signal.

11. A method of detecting a sleep stage, comprising:
receiving a radar signal and extracting a physiological signal from the radar signal;
performing fast Fourier transform on the physiological signal by using a first window to obtain a transformed signal;
obtaining a peak area ratio corresponding to the transformed signal according to a peak of the transformed signal;
generating a first prediction result of the sleep stage according to the peak area ratio by using a first machine learning model; and
outputting the first prediction result.

12. The method according to claim 11, further comprising:
extracting a sample of the radar signal by using a second window, wherein the second window is shorter than the first window;
inputting the sample into a second machine learning model to generate a second prediction result of the sleep stage; and
updating a portion of the first prediction result corresponding to the sample according to the second prediction result.

13. The method according to claim 12, wherein a step of updating the portion of the first prediction result corresponding to the sample according to the second prediction result comprises:
in response to the second prediction result indicating that a confidence level corresponding to a first sleep stage is greater than a threshold value, updating the portion of the prediction result to the first sleep stage.

14. The method according to claim 11, wherein a step of generating the first prediction result of the sleep stage according to the peak area ratio by using the first machine learning model comprises:
performing phase amplitude coupling on the physiological signal by using the first window to obtain a coupled signal; and
generating the first prediction result according to the coupled signal and the peak area ratio by using the first machine learning model.

15. The method according to claim 14, wherein a step of generating the first prediction result according to the coupled signal and the peak area ratio by using the first machine learning model comprises:
performing normalization on the peak area ratio to obtain the normalized peak area ratio;
performing the normalization on the coupled signal to obtain the normalized coupled signal;
calculating an average of the normalized peak area ratio and the normalized coupled signal to obtain an average signal; and
generating the first prediction result according to the average signal by using the first machine learning model.

16. The method according to claim 15, wherein a step of generating the first prediction result according to the average signal by using the first machine learning model comprises:
performing change point detection on the average signal to obtain a plurality of change points;
segmenting a sample from the average signal according to the change points; and
inputting the sample to the first machine learning model to generate the first prediction result.

17. The method according to claim 11, further comprising:
performing change point detection on the transformed signal to obtain the peak.

18. The method according to claim 17, wherein a step of obtaining the peak area ratio corresponding to the transformed signal according to the peak of the transformed signal comprises:
sampling the transformed signal according to a third window to obtain a first peak area, wherein a center of the third window corresponds to a frequency of the peak;
sampling the transformed signal according to a fourth window to obtain a second peak area, wherein the fourth window comprises the frequency of the peak; and
calculating a ratio of the first peak area and the second peak area to obtain the peak area ratio.

19. The method according to claim 18, further comprising one of the following:
setting the fourth window according to a preset time period; and
setting the fourth window according to the frequency of the peak, wherein a center of the fourth window corresponds to the frequency.

20. The method according to claim 11, wherein the physiological signal comprises at least one of a heart rate signal and a respiratory signal.
